(19) United States
(12) Patent Application Publication
(10) Pub. No.: US 2016/0220561 A1
(43) Pub. Date: Aug. 4, 2016

(54) PHARMACEUTICAL COMPOSITION OF BUPROPION AND NALTREXONE

(71) Applicant: Gavis Pharmaceuticals, Somerset, NJ (US)

(72) Inventors: James Garegnani, Somerset, NJ (US); Velayutham Kanniyappan, Somerset, NJ (US)

(73) Assignee: GAVIS PHARMACEUTICALS, Somerset, NJ (US)

(21) Appl. No.: 14/611,242

(22) Filed: Feb. 1, 2015

(51) Int. Cl. A61K 31/485; A61K 31/135

(52) U.S. Cl. CPC A61K 31/485; A61K 31/135

(57) ABSTRACT

A pharmaceutical composition of bupropion or salts thereof and naltrexone or salts thereof is provided. The composition comprises individual layers of bupropion or a pharmaceutically acceptable salt thereof and naltrexone or a pharmaceutically acceptable salt thereof separated by an intermediate layer. The intermediate layer is devoid of sugar. A method of manufacturing the composition is also provided.

### BACKGROUND OF THE INVENTION

(a) Field of the Invention

The present invention is directed to a pharmaceutical composition of bupropion or a pharmaceutically acceptable salt thereof and naltrexone or a pharmaceutically acceptable salt thereof. The invention is further directed to the use of said composition for affecting weight loss, suppressing appetite and/or treating obesity-related conditions in individuals. Additionally, the present invention provides a method of manufacture of said composition.

(b) Description of the Related Art

Obesity is a disorder characterized by the accumulation of excess fat in the body. Obesity has been recognized as one of the leading causes of disease and is emerging as a global problem. Increased instances of complications such as hypertension, non-insulin dependent diabetes mellitus, arteriosclerosis, dyslipidemia, certain forms of cancer, sleep apnea, and osteoarthritis have been related to increased instances of obesity in the general population.

In addition to those individuals who satisfy a strict definition of medical obesity, a significant portion of the adult population is overweight. These overweight individuals would also benefit from the availability of an effective weight-loss composition.

Various drugs are currently available for management of obesity or affecting weight loss including orlistat, lorcaserin, sibutramine, exenatide, pramlintide, amphetamine, and a combination of phentermine and topiramate.

U.S. Pat. No. 7,425,571 discloses a method of treating obesity by administering zonisamide in combination with bupropion.

U.S. Pat. Nos. 7,056,890 and 7,659,256 discloses a composition comprising phentermine and topiramate and its use for effecting weight loss.

Recently a fixed dose combination of bupropion and naltrexone has been approved in the United States for affecting weight loss, suppressing appetite and/or treating obesity-related conditions. The product is marketed in the United States by Takeda Pharmaceuticals under the brand name Contrave®. The product is in the form of a sustained release tri-layer tablet containing individual layers of bupropion and naltrexone separated by a sugar-containing intermediate layer.

Bupropion is used as an antidepressant. It has also been used either alone or in combination with other drugs as a smoking cessation aid. Bupropion hydrochloride is stable by itself under normal storage conditions, but can degrade in the presence of certain conventional excipients used in commercial formulations.

Naltrexone is an opioid antagonist. It is a synthetic congener of oxymorphone with no opioid agonist properties.

The bupropion and naltrexone combination has effects on two separate areas of the brain involved in the regulation of food intake: the hypothalamus (appetite regulatory center) and the mesolimbic dopamine circuit (reward system). The exact neurochemical effects of the combination leading to weight loss however are not fully understood.

U.S. Pat. Nos. 7,375,111; 7,462,626 and 8,722,085 disclose use of combinations of bupropion and naltrexone for the treatment of being overweight and obesity.

U.S. Pat. No. 8,815,889 discloses a method of treating insulin resistance by administering bupropion in combination with naltrexone.

U.S. Pat. Nos. 8,088,786 and 8,318,788 disclose a layered composition of bupropion and naltrexone in which the two drug layers are separated by an intermediate layer containing sugar as well as a method of affecting weight loss by administering said composition. Such composition requires complicated manufacturing processes, commercial manufacturing of which in turn consumes significant amount of time and is cost intensive.

There is still a need for improved pharmaceutical compositions comprising bupropion and naltrexone as well as an improved and relatively simple process for preparing such preparations.

### SUMMARY OF THE INVENTION

The present invention provides the following aspects, subject-matters and preferred embodiments, which respectively taken alone or in combination, further contribute to solving the object of the present invention.

As described in further detail below, advantageously the pharmaceutical composition comprises a first layer comprising bupropion or a pharmaceutically acceptable salt thereof, a second layer comprising naltrexone or a pharmaceutically acceptable salt thereof, and an intermediate layer disposed between said first and said second pharmaceutical layers. The intermediate layer is characterized in that it does not contain any sugar, i.e., the intermediate layer is free of sugar or essentially free of sugar. The composition is prepared using a simple and economical manufacturing process that is also suitable for large scale production.

The pharmaceutical composition may consist essentially of or consist of the first layer comprising bupropion or a pharmaceutically acceptable salt thereof, the second layer comprising naltrexone or a pharmaceutically acceptable salt thereof, and the intermediate layer disposed between the first and the second pharmaceutical layers.

In one aspect, the present invention provides a pharmaceutical composition comprising a first layer comprising bupropion or a pharmaceutically acceptable salt thereof, a second layer comprising naltrexone or a pharmaceutically acceptable salt thereof and an intermediate layer disposed between said first and said second pharmaceutical layers; wherein said intermediate layer is devoid of sugar. The individual drug layers further may comprise one or more release rate controlling agents in the form of a matrix. In an embodiment, the release rate controlling agent is provided in the form of a coating over the individual drug layer.

In another aspect, the pharmaceutical composition of the invention comprises bupropion or pharmaceutically acceptable salt thereof in a dose of about 30 mg to about 500 mg, and naltrexone or pharmaceutically acceptable salt thereof in a dose of about 1 mg to about 50 mg.

In an embodiment, the pharmaceutical composition of the invention comprises bupropion or pharmaceutically acceptable salt thereof in a dose of about 30 mg to about 300 mg, and naltrexone or pharmaceutically acceptable salt thereof in a dose of about 5 mg to about 50 mg.

In another aspect, bupropion or pharmaceutically acceptable salt thereof and/or naltrexone or pharmaceutically acceptable salt thereof in the composition exhibit sustained release.

In another aspect, each of bupropion or pharmaceutically acceptable salt thereof and naltrexone or pharmaceutically acceptable salt exhibits sustained release.

In another aspect, the present invention provides a trilayer tablet comprising a first layer comprising bupropion or a pharmaceutically acceptable salt thereof and one or more release rate controlling agents; a second layer comprising naltrexone or a pharmaceutically acceptable salt thereof; an intermediate layer disposed between said first and said second pharmaceutical layers; one or more release rate controlling agents; and one or more pharmaceutically acceptable excipients; wherein said first layer of bupropion or pharmaceutically acceptable salt thereof and second layer of naltrexone or pharmaceutically acceptable salt thereof exhibit sustained release.

In another aspect, the pharmaceutical composition is achieved by using a suitable dissolution release rate controlling agent of hydrophilic, lipophilic or inert character or a combination of several different rate controlling agents providing sustained release of the drugs.

In another aspect, the rate controlling agent in the composition is in an amount in the range of 10% to 60% w/w of the composition.

In another aspect, the invention provides a method of manufacturing a pharmaceutical composition comprising a first layer comprising bupropion or a pharmaceutically acceptable salt thereof, a second layer comprising naltrexone or a pharmaceutically acceptable salt thereof, and an intermediate layer disposed between said first and said second pharmaceutical layers. The method of manufacture of the composition comprises the steps of:
(a) preparing the first layer blend comprising bupropion or a pharmaceutically acceptable salt thereof and one or more pharmaceutically acceptable excipients;
(b) preparing the second layer blend comprising naltrexone or a pharmaceutically acceptable salt thereof and one or more pharmaceutically acceptable excipients;
(c) preparing the intermediate layer blend comprising one or more pharmaceutically acceptable excipients other than sugar; and
(d) compressing the blends together by maintaining the intermediate layer blend in between first and second layer to form the pharmaceutical composition.

In another aspect, the present invention provides the pharmaceutical composition as substantially described herein for use in the treatment of being overweight or obesity.

In another aspect, the present invention provides a method of affecting weight loss, suppressing appetite and/or treating obesity-related conditions in individuals. The method comprises administering the pharmaceutical composition as substantially described herein.

Still other aspects and advantages of the invention will be apparent from the following detailed description of the invention.

### DETAILED DESCRIPTION OF THE INVENTION

The invention provides for a pharmaceutical composition comprising a first layer of bupropion or a pharmaceutically acceptable salt thereof, a second layer of naltrexone or a pharmaceutically acceptable salt thereof, an intermediate layer disposed between said first and said second pharmaceutical layers, and pharmaceutically acceptable excipients in a single dosage form. The intermediate layer is characterized in that it is devoid of sugar. The drugs in the layers are constituted in the form of a homogeneous or heterogeneous matrix of drug, pharmaceutically acceptable excipients and, optionally, rate controlling agent. Preferably, no drug portion in the composition represents a coating.

The inventors have found that the composition according to the present invention eliminates the need of using sugars in the intermediate layer that was required to achieve a predictable dissolution profile of each individual drug that matches with in vivo release. Such composition also can be prepared by a simple manufacturing process.

A dissolution profile for a drug comprises the known dissolution rate and particular dissolution characteristics of the drug. A predictable dissolution profile for a specific drug allows for more accurate treatment of a given symptom. Predictable dissolution profiles for different drugs within a trilayer composition such as a tablet allow for coordinated treatment of multiple symptoms with a single pharmaceutical formulation.

It was also observed that the trilayer composition comprising a fixed dose combination of bupropion and naltrexone or pharmaceutically acceptable salts thereof is ingested by a patient, however, each individual layer and in turn, each drug dissolves as predicted by its individual dissolution profile.

A further advantage of the trilayer composition is that the dissolution of both drugs is not affected in the case where the composition is attached to the lining of the stomach and the dissolution for both drugs occurs in a predictable rate.

As mentioned above, having a trilayer composition is desirable for ease of administration of multiple pharmaceutical compositions within a composition or dosage form such as a tablet.

The pharmaceutical composition comprising two drug layers and an intermediate layer configured to dissolve in vivo to thereby to leave the two drug layers substantially intact. In preferred embodiments the dissolution rate of one or more of the separated drug layers is substantially similar to that of a singly compressed tablet comprising the same drug composition as that of the respective drug layer.

The terms “bupropion” and “naltrexone” denotes any pharmaceutical acceptable salts of bupropion and naltrexone. Within the meaning of the present invention, the term “bupropion” preferably refers to the active pharmaceutical ingredient “bupropion hydrochloride”. Within the meaning of the present invention, the term “naltrexone” preferably refers to the active pharmaceutical ingredient “naltrexone hydrochloride”. Within the context of the present specification, both bupropion and naltrexone are sometimes commonly referred to as “drugs”.

The term “controlled-release” is used herein in its ordinary sense and thus includes pharmaceutical compositions combined or coated with ingredients (e.g., rate controlling agents) to alter their dissolution profile. A “sustained-release” formulation is a type of controlled-release formulation in which ingredients have been added to a pharmaceutical composition such that the dissolution profile is extended over a longer period of time than that of an immediate release formulation comprising a similar pharmaceutical composition.

According to the invention, the pharmaceutical composition comprises a first layer of bupropion or a pharmaceutically acceptable salt thereof, a second layer of naltrexone or a pharmaceutically acceptable salt thereof, an intermediate layer disposed between said first and said second pharmaceutical layers, and pharmaceutically acceptable excipients; wherein the intermediate layer is devoid of sugar. Preferably, neither of the drugs represents a coating.

In an embodiment the at least one intermediate layer is a flat layer separating at least two drug layers. In a further embodiment the at least one intermediate layer has exposed edges. Exposed edges allow for fluid to contact and dissolve the at least one intermediate layer. In a further embodiment the composition comprises a coating covering the two drug layers and the at least one intermediate layer. The coating is configured to dissolve in vivo more or less uniformly over the two drug layers and the at least one intermediate layer such that the at least one intermediate layer is left exposed to the fluids that will dissolve the at least one intermediate layer in vivo.

In an embodiment the at least one intermediate layer is or comprises an impermeable membrane. In a further embodiment the at least one intermediate layer has a substantially higher dissolution rate than at least one of the drug layers. In a preferred embodiment the at least one intermediate layer dissolves in a nearly immediate fashion with respect to the dissolution of at least one of the drug layers.

The matrix further may comprise one or more release rate controlling agents. In an embodiment, the release rate controlling agent is provided in the form of a coating over the layer containing drug matrix.

The composition of the invention exhibits excellent storage stability when subjected to storage at 40° C. and 75% relative humidity for a period of 3 months. The dosage form retains at least 95% of the total potency of bupropion and naltrexone upon storage.

The composition of the invention comprises individual layers of bupropion, naltrexone or a pharmaceutically acceptable salt thereof, at least one release rate controlling agent, and one or more pharmaceutically acceptable excipients.

The pharmaceutical composition of the invention comprises bupropion or pharmaceutically acceptable salt thereof in a dose of about 30 mg to about 500 mg and naltrexone or pharmaceutically acceptable salt thereof in a dose of about 1 mg to about 50 mg. Preferably, the composition comprises bupropion or pharmaceutically acceptable salt thereof in a dose of about 30 mg to about 300 mg and naltrexone or pharmaceutically acceptable salt thereof in a dose of about 5 mg to about 50 mg.

The first layer of bupropion or pharmaceutically acceptable salt thereof and second layer of naltrexone or pharmaceutically acceptable salt thereof in the composition both exhibit sustained release. In an embodiment, the first layer of bupropion or pharmaceutically acceptable salt thereof exhibits sustained release or the second layer of naltrexone or pharmaceutically acceptable salt exhibits sustained release.

The composition of bupropion or pharmaceutically acceptable salt thereof and naltrexone or pharmaceutically acceptable salt thereof is provided and administered in a single oral dosage form. Suitable dosage forms include, but are not limited to the form of a tablet, pill, or capsule. Preferably the dosage form is a trilayer tablet.

Sustained release of the drugs from the pharmaceutical composition is achieved by using a suitable dissolution release rate controlling agent of hydrophilic, lipophilic or inert character or a combination of several different release rate controlling agents providing controlled release of the drugs.

Suitable release rate controlling agents, by way of example and without limitation, may be selected from the group consisting of hydrophilic agents, lipophilic agents and inert agents. The hydrophilic agents are selected from the group of pharmaceutical excipients which generate a gel upon contact with water, including cellulose derivatives such as hydroxypropyl methyl cellulose, hydroxyethyl cellulose, hydroxypropyl cellulose, methyl cellulose and the like; noncellulose polysaccharides such as galactomannanes, guar gum, carob gum, gum arabicum, alginates, pectins, and the like; polyvinylpyrrolidone; polyvinylacetate polymers and copolymers; acrylic acid polymers and copolymers, polyethylene oxide and mixtures thereof. The lipophilic agents are selected from the group consisting of waxes such as white wax, bees wax, carnauba wax and the like; fatty acids and alcohols such as stearic acid, palmitic acid, lauric acid and the like, and cetyl alcohol, cetostearyl alcohol, stearyl alcohol and the like; fatty acids esters such as monostearates of propylene glycol and fatty acid esters of sucrose, sucrose distearate and the like; glycerides such as mono-, di- or triglycerides, e.g., palmitin, stearin, behenic, laurin, myristin, hydrogenated vegetable, castor, cottonseed oils, glyceril behenate and the like; and mixtures thereof. The inert agents are selected from the group consisting of thermoplastic polymers that are insoluble and indigestible in the gastrointestinal fluids, such as polyvinyl chloride, polyethylene, vinyl acetate/vinyl chloride copolymers, polymethylmethacrylates, polyamides, silicones, ethyl cellulose, polystyrene, and mixtures thereof. The release rate controlling agent in the composition is preferably present in an amount in the range of 10% to 60% w/w of the composition.

The pharmaceutical composition may comprise one or more pharmaceutically acceptable excipients selected from the group consisting of binding agents, fillers, filler-binders, disintegrants, lubricants, sweeteners, glidants, flavourings and colouring agents.

The pharmaceutical composition may be configured in various shapes and sizes for ease of administration to a patient. Manufacture of the pharmaceutical composition configured in tablets comprises steps known in the art. For example, tablets may be prepared through wet-granulation, dry-granulation or direct compression. Layered pharmaceutical formulations may be configured in a tablet form in a similar manner. To manufacture each drug layer, one or more drugs are obtained in, for example, a crystalline, amorphous or powdered form, and mixed with or without diluents and/or excipients into a solid with pressure. The solid drug layer is added with other drug layers and/or intermediate layers and configured in a desired tablet geometry with pressure.

In an embodiment, the method of manufacturing the pharmaceutical composition comprises the steps of:
(a) preparing the first layer blend comprising bupropion or a pharmaceutically acceptable salt thereof and one or more pharmaceutically acceptable excipients;
(b) preparing the second layer blend comprising naltrexone or a pharmaceutically acceptable salt thereof and one or more pharmaceutically acceptable excipients;
(c) preparing the intermediate layer blend comprising one or more pharmaceutically acceptable excipients other than sugar; and
(d) compressing the blends together by maintaining the intermediate layer blend in between first and second layer to form the pharmaceutical composition.

The invention further provides the pharmaceutical composition as substantially described herein for use in the treatment of being overweight or obesity.

The invention further provides a method of affecting weight loss, suppressing appetite and/or treating obesity-related conditions in individuals comprising administering the pharmaceutical composition as substantially described herein.

### EXAMPLE 1

### Naltrexone Hydrochloride and Bupropion Hydrochloride Extended-Release Tablet

TABLE 1

| | | |
|---|---|---|
| | | Quantity |
| Sr. | | gm/5000 |
| No. | Ingredients | Tablets |

Layer 1

| | | |
|---|---|---|
| 1 | Naltrexone HCl | 40.00 |
| 2 | Hydroxyethyl Cellulose | 112.50 |
| 3 | Hydroxypropyl Cellulose | 225.00 |
| 4 | Hypromellose K100 | 75.00 |
| 5 | Lactose Monohydrate | 36.25 |
| 6 | Magnesium Stearate | 11.25 |

Layer 2

| | | |
|---|---|---|
| 7 | Microcrystalline Cellulose | 400.00 |
| 8 | Pregelatinized Corn Starch | 100.00 |
| 9 | Sodium Stearyl Fumarate | 5.00 |

Layer 3

| | | |
|---|---|---|
| 10 | Bupropion HCl | 450.00 |
| 11 | Hydroxyethyl Cellulose | 75.00 |
| 12 | Hydroxypropyl Cellulose | 150.00 |
| 13 | Hypromellose K100 | 50.00 |
| 14 | Lactose Monohydrate | 20.00 |
| 15 | Magnesium Stearate | 7.50 |

Procedure:

Layer 1: Naltrexone HCl, Hydroxyethyl Cellulose, Hydroxypropyl Cellulose, Hypromellose K100, Lactose Monohydrate and half of the Magnesium Stearate were screened and blended together. The mixture was passed through a roller compactor and milled. The milled blend was mixed with remaining amount of Magnesium Stearate.

Layer 2: Microcrystalline Cellulose, Pregelatinized Corn Starch and Sodium Stearyl Fumarate were screened and blended together.

Layer 3: Bupropion HCl, Hydroxyethyl Cellulose, Hydroxypropyl Cellulose, Hypromellose K100, Lactose Monohydrate and half of the Magnesium Stearate were screened and blended together. The mixture was passed through a roller compactor and milled. The milled blend was mixed with the remaining amount of Magnesium Stearate.

The three layers were then compressed together into a tablet using a multi-layer tablet press maintaining Layer 2 in-between Layer 1 and Layer 3.

### EXAMPLE 2

### Naltrexone Hydrochloride and Bupropion Hydrochloride Extended-Release Tablet

TABLE 2

| | | |
|---|---|---|
| | | Quantity |
| Sr. | | gm/5000 |
| No. | Ingredients | Tablets |

Layer 1

| | | |
|---|---|---|
| 1 | Naltrexone HCl | 40.00 |
| 2 | Hypromellose K100 | 184.00 |
| 3 | Carbomer Homopolymer (Type A) 971P | 161.00 |
| 4 | Carbomer Homopolymer (Type A) 71G | 23.00 |
| 5 | Lactose Monohydrate | 83.95 |
| 6 | Colloidal Silicone Dioxide | 1.15 |
| 7 | Magnesium Stearate | 6.90 |

Layer 2

| | | |
|---|---|---|
| 8 | Microcrystalline Cellulose | 475.00 |
| 9 | Sodium Starch Glycolate | 25.00 |
| 10 | Magnesium Stearate | 5.00 |

Layer 3

| | | |
|---|---|---|
| 11 | Bupropion HCl | 450.00 |
| 12 | Hypromellose K100 | 75.00 |
| 13 | Hydroxypropyl Cellulose | 150.00 |
| 14 | Carbomer Homopolymer (Type A) 971P | 50.00 |
| 15 | Lactose Monohydrate | 20.00 |
| 16 | Magnesium Stearate | 7.50 |

Procedure:

Layer 1: Naltrexone HCl, Hypromellose K100, Carbomer Homopolymer (Type A) 971 P, Carbomer Homopolymer (Type A) 71G, Lactose Monohydrate and half of the Magnesium Stearate were screened and blended together. The mixture was passed through a roller compactor and milled. The milled blend was mixed with Colloidal Silicone Dioxide and the remaining amount of Magnesium Stearate.

Layer 2: Microcrystalline Cellulose, Pregelatinized Corn Starch and Sodium Stearyl Fumarate were screened and blended together.

Layer 3: Bupropion HCl, Hydroxyethyl Cellulose, Hypromellose K100, Carbomer Homopolymer (Type A) 971 P, Lactose Monohydrate and half of the Magnesium Stearate were screened and blended together. The mixture was passed through a roller compactor and milled. The milled blend was mixed with the remaining amount of Magnesium Stearate.

Three layers were then compressed together into a tablet using a multi-layer tablet press maintaining Layer 2 in-between Layer 1 and Layer 3.

1. A pharmaceutical composition comprising:
a first layer comprising bupropion or a pharmaceutically acceptable salt thereof;
a second layer comprising naltrexone or a pharmaceutically acceptable salt thereof;
an intermediate layer comprising a lubricant disposed between said first and said second layers; and one or more pharmaceutically acceptable excipients; wherein the intermediate layer is devoid of sugar.

2. The pharmaceutical composition of claim 1, wherein neither of the first and second layers represents a coating.

3. The pharmaceutical composition of claim 1, wherein said first layer comprising bupropion or a pharmaceutically acceptable salt thereof and second layer comprising naltrexone or a pharmaceutically acceptable salt thereof exhibit sustained release of the bupropion or naltrexone, respectively.

4. The pharmaceutical composition of claim 1, wherein the first layer and/or second layer comprises one or more release rate controlling agents.

5. The pharmaceutical composition of claim 1, wherein the first layer and/or the second layer is/are coated with one or more release rate controlling agents.

6. The pharmaceutical composition of claim 4, wherein said release rate controlling agent is selected from the group consisting of hydroxypropyl methyl cellulose, hydroxyethyl cellulose, hydroxypropyl cellulose, methyl cellulose, galactomannanes, guar gum, carob gum, gum arabicum, alginates, pectins, polyvinylpyrrolidone; polyvinylacetate polymers and copolymers, acrylic acid polymers and copolymers, polyethylene oxide, white wax, bees wax, carnauba wax, stearic acid, palmitic acid, lauric acid, cetyl alcohol, cetostearyl alcohol, stearyl alcohol, monostearates of propylene glycol and fatty acid esters of sucrose, sucrose distearate, hydrogenated vegetable oil, glyceril behenate, polyvinyl chloride, polyvinyl alcohol, polyethylene, vinyl acetate/vinyl chloride copolymers, polymethylmethacrylates, polyamides, silicones, ethyl cellulose, polystyrene, and mixtures thereof.

7. The pharmaceutical composition of claim 4, wherein said release rate controlling agent is present in an amount in the range of 10% to 60% w/w of the composition.

8. The pharmaceutical composition of claim 1, wherein said bupropion or pharmaceutically acceptable salt thereof is in a dose of about 30 mg to about 500 mg, and said naltrexone or pharmaceutically acceptable salt thereof is in a dose of about 1 mg to about 50 mg.

9. The pharmaceutical composition of claim 1, wherein said bupropion or pharmaceutically acceptable salt thereof is in a dose of about 30 mg to about 300 mg, and said naltrexone or pharmaceutically acceptable salt thereof is in a dose of about 5 mg to about 50 mg.

10. The pharmaceutical composition of claim 1, wherein said composition is in the form of a bilayer tablet.

11. A method of manufacturing the pharmaceutical composition of claim 1, which method comprises the steps of:
(a) preparing the first layer blend comprising bupropion or a pharmaceutically acceptable salt thereof and one or more pharmaceutically acceptable excipients;
(b) preparing the second layer blend comprising naltrexone or a pharmaceutically acceptable salt thereof and one or more pharmaceutically acceptable excipients;
(c) preparing the intermediate layer blend comprising one or more pharmaceutically acceptable excipients other than sugar; and
(d) compressing the blends together by maintaining the intermediate layer blend in between the first layer and the second layer to form the pharmaceutical composition.

12. A trilayer tablet comprising:
a first layer comprising bupropion or a pharmaceutically acceptable salt thereof;
a second layer comprising naltrexone or a pharmaceutically acceptable salt thereof;
an intermediate layer disposed between said first and said second pharmaceutical layers;
and one or more pharmaceutically acceptable excipients; wherein the intermediate layer comprises a lubricant and is devoid of sugar.

13. The pharmaceutical composition of claim 1 for use in the treatment of overweight or obesity.

14. A method of affecting weight loss, suppressing appetite and/or treating obesity-related conditions in an individual comprising administering the pharmaceutical composition of claim 1 to said individual.

15. The pharmaceutical composition of claim 1, wherein the lubricant comprises magnesium stearate.

16. The pharmaceutical composition of claim 1, wherein the lubricant comprises sodium stearyl fumarate.

17. The pharmaceutical composition of claim 1, wherein the lubricant comprises approximately 1% by weight of the intermediate layer.

18. The pharmaceutical composition of claim 1, wherein the intermediate layer further comprises microcrystalline cellulose and a starch.

19. The pharmaceutical composition of claim 18, wherein the starch comprises sodium starch glycolate or pregelatinized starch.

20. The pharmaceutical composition of claim 12 wherein the lubricant comprises magnesium stearate or sodium stearyl fumarate and the lubricant is present at approximately 1% by weight of the intermediate layer.
